(19) United States
(12) Patent Application Publication
(10) Pub. No.: US 2004/0058402 A1
(43) Pub. Date: Mar. 25, 2004

(54) Method for purifying the helicobacter adhesin-like protein a (alpa)

(76) Inventors: Laurence Fourrichon, Lucenay (FR); Ling Lissolo, Marcy l'Etoile (FR); Olivier Pitiot, Saint Genis les Ollieres (FR)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/470,780

(22) PCT Filed: Jan. 30, 2002
(86) PCT No.: PCT/FR02/00355

(30) Foreign Application Priority Data
Feb. 5, 2001 (FR) 01/01499

(51) Int. Cl. G01N 33/554; G01N 33/569; C07K 14/195

(52) U.S. Cl. 435/7.32; 530/417

(57) ABSTRACT

The invention concerns a method for purifying the Helicobacter adhesin-like protein A (AlpA) which consists in: (i) contacting an AlpA preparation and 2.5 to 3.5 M of guanidine with a hydrophobic interaction chromatography material, so that the AlpA is adsorbed on the material; and (ii) eluting the AlpA with a solution containing 3.5 to 4.5 M of guanidine. The AlpA preparation to be purified can be in particular derived from an E. coli culture capable of expressing AlpA in a high-level recombinant form, rAlpA being in the form of inclusion bodies, the latter being recovered and solubilized in the presence of guanidine, and optionally ammonium sulphate-precipitated for the purpose of preliminary purification. The hydrophobic interaction chromatography can be followed up by an anion exchange chromatography in the presence of 8 M of urea.

The subject of the invention is a method for purifying the Helicobacter AlpA protein which possesses the characteristics required for use on an industrial scale.

Helicobacter is a bacterial genus characterized by gram-negative helical bacteria. Several species colonize the gastrointestinal tract of mammals. There may be mentioned in particular H. pylori, H. heilmanii, H. felis and H. mustelae. Although H. pylori is the species most commonly associated with human infections, in some rare cases, it has been possible to isolate H. heilmanii and H. felis from humans. A Helicobacter-type bacterium Gastrospirillum hominis has also been described in humans.

Helicobacter infects more than 50% of the adult population in developed countries and nearly 100% of that in developing countries, which makes it one of the predominant infection agents worldwide.

H. pylori is found exclusively to date at the surface of the mucous membrane of the stomach in humans and more particularly around the crater lesions of gastric and duodenal ulcers. This bacterium is currently recognized as the etiological agent of entral gastritis and appears as one of the cofactors required for the development of ulcers. Moreover, it appears that the development of gastric carcinomas may be associated with the presence of H. pylori.

It therefore appears highly desirable to develop a vaccine in order to prevent or treat Helicobacter infections.

To date, several Helicobacter proteins have already been proposed as a vaccine antigen and among these, the lipoprotein AlpA (Adhesin-like lipoprotein A) of membrane origin. AlpA and the gene encoding the latter were initially disclosed in WO 96/41880. A method of production and purification which makes it possible to prepare batches of pharmaceutical grade therefore remained to be developed.

Thus, the main objective of the present invention is to provide a simple and robust method which can be used on an industrial scale. This method should especially make it possible to produce a large quantity of antigen without having to make expensive investments.

E. coli is par excellence the bacterium used as host organism in order to produce all sorts of heterologous proteins by the recombinant route. When these proteins are not soluble or excreted in one way or another, they can accumulate in the form of inclusion bodies. These inclusion bodies are intracytoplasmic corpuscles specific to E. coli. The appearance of these inclusion bodies is promoted by a high level of expression (overexpression). The purification of the recombinant proteins from the inclusion bodies requires especially that these inclusion bodies are solubilized beforehand. This solubilization is customarily obtained with a chaotropic agent (urea, guanidine) or a detergent.

The recombinant protein rAlpA, deliberately produced in nonlipidated form in E. coli, still retains a high hydrophobicity. This high intrinsic hydrophobicity renders its purification difficult by conventional means. Furthermore, the rAlpA protein is found in the inclusion bodies of E. coli and in the presence of a chaotropic agent in a high concentration is practically obligatory throughout the purification.

For a solubilization at neutral pH, the high hydrophobicity of AlpA requires a high concentration of guanidine; this agent therefore appears as an essential factor in the operation for solubilizing inclusion bodies. Nevertheless, this agent which suppresses the ionic and hydrophobic interactions, is usually not widely used for purification by chromatography and therefore ought to be eliminated.

In general, hydrophobic interaction chromatographies (HIC) are commonly used in order to purify proteins having a marked hydrophobic character. However, as mentioned above, when such a protein is present in a medium with a high concentration of guanidine, the use of this type of chromatography becomes highly problematic because the presence of guanidine should normally prevent adsorption. Indeed, a very large number of proteins which can absorb under conventional conditions on a HIC support are thought to be eluted at a high guanidine concentration.

However, as regards AlpA, it has now been found that a very narrow guanidine concentration window existed which allowed AlpA to be maintained in soluble form and its adsorption onto an HIC support. This window is between about 2.5 and 3.5 M.

Accordingly, the subject of the invention is a method for purifying Helicobacter, e.g. H. pylori, adhesin-like protein A (AlpA), according to which (i) a preparation containing AlpA and 2.5 to 3.5, M guanidine is brought into contact with a hydrophobic interaction chromatography material such that AlpA adsorbs onto the material and (ii) AlpA is eluted with a solution containing 3.5 to 4.5 M guanidine.

Preferably, the preparation brought into contact with the chromatography material contains 3 M guanidine and AlpA is eluted with 4 M guanidine at neutral pH (6-8; e.g. 6.5-7.5).

Thus, as mentioned above, a preparation containing AlpA in the presence of guanidine may be especially derived from a method of production according to which (a) a Gram-negative bacterium, especially E. coli, capable of expressing AlpA in recombinant form, is cultured, (b) the bacterial cells derived from the culture are recovered and they are lysed, (c) the inclusion bodies contained in the cells are recovered, they are washed and they are solubilized, and (d) optionally, the inclusion bodies are precipitated, the supernatant is removed and the precipitate is solubilized.

Further details are given below. The bacteria harvested after culture are first of all lysed. The cellular lysis may be carried out in various ways, in particular by microfluidization. According to a particular embodiment, it is also possible to treat the cells with benzonase during the lysis, so that the E. coli DNA is digested. The inclusion bodies are then subjected to successive washes, preferably in a buffer containing a high concentration of salt so as to eliminate the nucleic acids and to dissociate as much as possible the rAlpA from the contaminants; in an appropriate manner, the buffer may be a sodium buffer, e.g. a buffer containing 0.5 M NaCl. The inclusion bodies are finally dissolved in highly concentrated guanidine, e.g. 5.5 to 6.5 M, preferably 6 M, guanidine. According to a particular embodiment, a portion of the contaminant bacterial proteins (e.g. from E. coli) is at this stage preferably removed by selectively precipitating rAlpA by “salting out” using ammonium sulfate. Preferably, the ammonium sulfate is 0.4 to 0.6 M, e.g. 0.5 M, ammonium sulfate. The precipitate containing rAlpA is again solubilized in highly concentrated guanidine, e.g. 5.5 to 6.5 M, preferably 6 M, guanidine. In order to use an hydrophobic interaction chromatography, the preparation is diluted ½ and then loaded onto an appropriate column.

The hydrophobic interaction chromatography may be carried out in a conventional manner using supports such as Sepharose™ (Pharmacia) and Fractogel™ (Merck) or supports equivalent to the latter, such as those found at Tosohaas or Biorad. The ligand may be a butyl, octyl or phenyl. The HIC allows in particular the separation of the AlpA protein from its degradation products.

According to a preferred embodiment, the eluate derived from the HIC may be concentrated in various ways and in particular by ultrafiltration, it is also advisable to remove the guanidine which is a toxic product. To this end, it is possible to use a diafiltration, e.g. tangential diafiltration, step, against several volumes of neutral buffer eluate containing urea having a molarity greater than or equal to 4 M, preferably greater than or equal to 6 M, preferably 8 M urea; the urea being present to ensure that rAlpA is maintained in a soluble form. According to an advantageous embodiment, 6 to 10 volumes of 7.5 to 8.5 M urea are used.

Finally, to complete the purification, it is possible to carry out a “polishing” by ion-exchange chromatography, in particular by anion-exchange chromatography in order to remove the residual contaminants from E. coli. According to an appropriate embodiment, the latter step is carried out in the presence of 8 M urea. The rAlpA preparation derived from the diafiltration is then applied to the anion exchange chromatography material; the contaminants are adsorbed onto this material while rAlpA does not adsorb and is found in the filtrate (also called direct eluate). The presence of 8 M urea is essential for the following reasons: AlpA is a positively charged protein (very high pI) and should therefore not normally adsorb onto anion-exchange chromatography material. However, its hydrophobic character is so marked that it would still adsorb if the 8 M urea was absent from the preparation.

In this regard, the invention also relates to a method for purifying AlpA according to which (i) a preparation containing AlpA and 7.5 to 8.5 M, preferably 8 M, urea is brought into contact with anion-exchange chromatography material such that the contaminants adsorb onto this material and (ii) AlpA is recovered in the filtrate.

The anion-exchange chromatography may be carried out in a conventional manner using supports such as Sepharose™ (Pharmacia) and Fractogel (Merck) or supports equivalent to the latter, such as those found at Tosohaas or Biorad. The ligand may be a tertiary amine such as DEAE (diethylaminoethyl) or a quaternary amine such as Q (quaternary, hydroxypropyldiethylaminoethyl) and TMAE (trimethylaminoethyl).

The method for purifying AlpA which is the subject of the present invention makes it possible, in its universality, to reach an unpresidented level of purity for the protein considered, in particular taking into account the large quantity initially treated. Accordingly, the invention also relates to:

The AlpA protein, characterized by a degree of purity greater than or equal to 90% of monomer, as measured by SDS-PAGE, Coomassie blue staining and densitometric reading. The remaining material other than the monomeric form consists of AlpA protein in multimeric form. The overall level of purity being greater than or equal to 99%.

A preparation containing at least 5 g, preferably at least 10 g, most preferably at least 15 g of the AlpA protein, the latter being characterized by a level of purity greater than or equal to 90% of monomer, as measured by SDS-PAGE, Coomassie blue staining and densitometric reading. Quite obviously, this precipitation should constitute a batch, that is to say should have been obtained from a single culture and an individual implementation of a method of purification.

Alternatively, it was also found that the inclusion bodies could be solubilized by replacing the guanidine with 7.5 to 8.5, preferably 8 M, urea in a buffer at basic, pH, preferably greater than or equal to 10.5, e.g. at pH 11. Advantageously, a phosphate buffer is used. Once the solubilization has been achieved, the molarity of the urea is reduced to between 4.5 and 5.5 M, e.g. 0.5 M, as well as the pH, to around neutrality, e.g. at 7.5. This may be carried out by diluting with a Tris buffer. An HIC is then carried out in which AlpA is diluted in 7.5 to 8.5 M, e.g. 8 M, urea.

Accordingly, the subject of the invention is also a method for purifying the Helicobacter adhesin-like protein A (AlpA) according to which (i) a preparation containing AlpA and 4.5 to 5.5 M urea is brought into contact with a hydrophobic interaction chromatography material such that AlpA adsorbs onto the material and (ii) AlpA is eluted with a solution containing 7.5 to 8.5 M urea.

### EXAMPLE

Expression System

An inducible expression system was constructed in order to produce the Helicobacter pylori AlpA protein in E. coli. The vector which was used was derived from the plasmid pET28c (Novagen). This vector comprises:

an expression cassette under the control of the T7 bacteriophage promoter,

a polylinker intended for the cloning of the genes of interest downstream of the promoter,

a transcriptional terminator also derived from the T7 bacteriophage, and

a Kanamycin-resistance gene.

The promoter is positively regulated in the presence of T7 RNA polymerase.

For the purpose of expression, various genetically modified E. coli strains may be used, among which the strains BL21 λ DE3 (Studier, F. W. et al 1990 Meth. Enzymol 185, 60-69.). These strains contain the gene encoding the T7 phage RNA polymerase under the control of the lac UV5 promoter, inducible by addition of IPTG. Furthermore, the BL21λDE3 strain is deficient for the OmpT and Lon protease activities.

A DNA fragment of 1.6 Kb containing the sequence encoding the mature AlpA protein was obtained by PCR amplification using the strain Helicobacter pylori X47-2 (ORV 2001) as source of DNA. The NcoI and XhoI restriction sites used for the cloning are included in the 5′ (HP3 Nco. C-) and 3′ HP3 (Xho) PCR primers respectively. The lipidation site [Cysteine codon (TGC codon)] was replaced by an ATG initiation codon (Met). The PCR-amplified fragment was first cloned into the shuttle vector Topo TA (Invitrogen), and then transferred into pET28c using the NcoI and XhoI sites.

The PCR amplification conditions were as follows: 97° C./30 s; 55° C./1 min; 72° C./50 s; Vent DNA polymerase—25 cycles.

Primer 5′:5′CATGCCATGGCTAGCATAAGTTATGCCGAA3′
Primer 3′:5′CCCTCGAGCCTTTCTTAGAATGAATACCCATA3′

The NcoI and XhoI sites are indicated in italics and the ATG initiation and TAA termination (opposite strand) codons are in underlined bold characters.

Preculture and Culture

2 liter Erlenmeyer flasks each containing 500 ml of modified 2× Luria Broth (LB) medium (peptone replaced with 30 g/L yeast extract) supplemented with 50 μg/mL of kanamycin, are inoculated with 500 μL of a batch of E. coli BL21DE3/pMHp3.1 inoculum and are incubated for 15-18 h at 37° C., with stirring at 175 rpm.

This preculture serves to inoculate a 30 L fermenter (B Braun) containing the SKYE 4 medium (for one liter: 40 g yeast extract; 3 ml 1 M MgCl₂; 530 mg K₂SO₄; 1 g NaCl; 844 g K₂HPO₄) supplemented with 44 g/l glucose monohydrate and 50 μg/ml kanamycin. The inoculum corresponds to 5% of the total volume. The culture parameters are the following: pH: 7.00; regulation 10% H₃PO₄ and 28% NH₄OH; temperature: 37° C.; initial stirring: 200 rpm; initial air flow rate: 1 l/l of culture/minute; PO2: 30%, regulation (1) stirring 200 rpm to 700 rpm (2) air flow-rate 20% to 60% (3) O₂ flow rate 0% to 100%.

The culture is continued for more than 3 h. When the OD at 600 nm is between 25 and 35, the expression of AlpA is induced by adding the volume of IPTG (at 200 mM) necessary to obtain a final concentration equal to 1 mM. After 2 h of induction, the culture is immediately cooled. The cells are harvested by centrifugation for 20 min at 7000 g and the pellets stored at −35° C.

Preparation of the Inclusion Bodies

Suspension of the Microorganisms and Treating of the Wet Pellet

Five hundred grams of cellular pellets (wet weight) (corresponding to about 5 L of culture) are thawed overnight at 5±3° C. and suspended in 50 mM Tris buffer pH 8.0 previously cooled to 5±3° C. in 7 ml of buffer per g of cell pellet, that is 3.75 L per 500 g. The expected volume of this suspension is 4.25 L. This suspension is homogenized with an Ultraturrax for 2 to 3 cycles of 30 to 60 sec and at a temperature maintained at 5±3° C. on an ice bath.

A solution of benzonase at 2.5 IU/μL is freshly prepared from a stock solution at 250 IU/μL stored at −20° C., that is 20 μL+1980 μL of 50 mM Tris buffer pH 8.0. 1700 μL of this solution are then added to 42.5 ml of MgCl₂ at 100 mM. The whole of this preparation is poured into the suspension of microorganism. The mixture is incubated at 5±3° C. with magnetic stirring for at least 30 minutes.

Breaking of the Cells

The cells thus homogenized are broken using a PANDA cell disintegrator at a fixed pressure of 1000 bar, with 3 breaking cycles and at a temperature not exceeding 15° C., using a cooling system (cryostat regulated at +5° C.). The ratio OD before breaking/OD after the last breaking should be ≧4.5.

Washing of the Inclusion Bodies

The suspension of inclusion bodies after 3 breaking cycles is centrifuged at 10 000 g for 30 min at 0.5±3° C. to remove contaminants (DNA, RNA and lipopolysaccharides, and the like).

After centrifugation, the pellet is recovered and then resuspended in 4.25. L of 50 mM sodium phosphate buffer containing 0.5 M, NaCl; pH 7.0, cooled to 5±3° C. The protein concentration is about 8 g/L. This suspension is homogenized using an Ultraturrax on an ice bath so as to maintain the suspension at 5±3° C. and then kept magnetically stirred for 30 min at, 5±3° C. The suspension thus washed is centrifuged at 10 000 g for 30 min and 5±3° C.

The washing operation is repeated twice. 110 g of washed inclusion bodies, that is about 0.3 g of proteins per gram of washed inclusion bodies, are thus obtained. Storage is possible at −20° C.

Purification from Inclusion Bodies

Solubilization of the Inclusion Bodies

The inclusion bodies are then solubilized in 50 mM Tris buffer containing 6 M Guanidine pH 7.5. The final protein concentration to be obtained is 20 g/L. The volume of buffer to be added is therefore about 1.65 L.

This suspension is homogenized using an Ultraturrax at 16 000 rpm and then with magnetic stirring until complete solubilization is obtained, at a temperature maintained at 22±3° C. The mixture is centrifuged at 10 000 g for 30 minutes at 22±3° C. in order to clarify the solution; the solution is then filtered in a sterile manner on Spiral Cap 0.8/0.2 μm. The volume of the supernatant filtered is about 1.6 L.

Precipitation with Ammonium Sulfate

The supernatant is diluted by half with a solution of 1 M ammonium sulfate, 50 mM Tris pH 7.5, with magnetic stirring. The solution thus obtained is added over 30 minutes with the aid of a peristaltic pump at a slow and uniform flow rate in order to obtain a solution with 10 g/L of proteins containing 0.5 M (NH₄)₂SO₄ final. The medium is kept magnetically stirred for 30 minutes at 22±3° C. The incubation is then continued without stirring overnight (15 hours) at laboratory temperature to allow ripening of the precipitate.

The next morning, the mixture is centrifuged for 30 min at 10 000 g and 22±3° C. The precipitate is collected and then solubilized at 10 g/L of proteins with 50 mM Tris buffer containing 6 M Guanidine pH 7.5, with magnetic stirring (the expected volume after solubilization is close to 3.2L). This suspension is homogenized using an Ultraturrax at 16 000 rpm and then with magnetic stirring until complete solubilization is obtained, at a temperature maintained at 22±3° C.

Hydrophobic Interaction Chromatography

The suspension is then diluted ½ by adding 50 mM Tris buffer containing 1.4 M NaCl pH 7.5, with magnetic stirring. This buffer is added over 30 minutes using a peristaltic pump at a slow and uniform flow rate in order to obtain a solution with 5 g/L of proteins containing 50 mM Tris, 0.7 M NaCl, 3 M Guanidine pH 7.5 final. The medium is kept magnetically stirred for 15 minutes at 22±3° C.

The mixture is centrifuged at 10 000 g for 30 minutes at 22±3° C. minutes in order to clarify the solution; it is then filtered in a sterile manner on Supor Cap. 0.8/0.2 μm. The volume of the filtered supernatant is about 6 L.

The column used possesses the following characteristics:

Column (Hi Prep butyl Sepharose FF Pharmacia—Amersham) having a diameter of 20 cm and a surface area of 314 cm²; gel height close to 26 cm; gel volume close to 8 L in conformity with the capacity of 3.2 g of proteins/mL of gel.

The column is prepared as follows:

The gel is washed and then equilibrated at a flow rate of close to 60 mL/cm².h (19 L/h) with. (i) 5 column volumes of ultrafiltered water, and then with (ii) 3 column volumes of 50 mM Tris buffer containing 3 M guanidine, 0.7 M NaCl pH 7.5. The conductivity and pH values of the buffer at the outlet of the column should correspond to the following specifications for use: conductivity at 20° C.=135 ms/cm±15%, and (ii) pH=7.5±0.3.

The chromatography is carried out as follows:

The column is loaded, by injecting the filtrate (corresponding to 22-25 g of total proteins) at a flow rate in the region of 60 mL/cm².h (19 L/h). The column is then rinsed with 50 mM Tris buffer containing 3 M Guanidine, 0.7 M NaCl pH 7.5 at a flow rate in the region of 90 mL/cm².h (28 L/h) until there is a return to the baseline (2 column volumes). AlpA is then eluted in 50 mM Tris buffer containing 4 M Guanidine pH 7.5 at a flow rate of 90 ml/cm².h (28 L/h) until there is a return to the base line (1 column volume). The volume of eluate is about 8 L.

Concentration and Difiltration on 30 kDa

The ultrafiltration model is prepared as follows:

4 Ultrafiltration cassettes made of polyether sulfone, having a cut-off of 30 kDa, each having a surface area in the region of 5000 cm² (that is a total surface area of 20 000 cm²), are used to concentrate about 20 g of proteins. These cassettes are washed with 0.5 N NaOH for 1 hour and then rinsed with ultrafiltered water.

They are then equilibrated with 50 mM Tris buffer containing 8 M Urea pH 7.5 for 5 min using a pump with an output in the region of 80 L/h.

The rAlpA eluate is concentrated in 50 mM Tris buffer containing 4 M Guanidine pH 7.5, around 5 g/L of proteins (concentration factor 2.5). The ultrafiltration conditions are the following: (i) inlet pressure in the region of 1 bar, and (ii) flow rate of the ultrafiltrate half that of the retentate. The volume of the concentrated eluate is about 3.5 L.

The concentrated rAlpA eluate is diafiltered against 10 volumes of 50 mM Tris containing 8 M urea pH 7.5 (35 L) under the same conditions as the concentration, at a flow rate of about 30 L/h, until there is obtained:

(i) a conductivity at 20° C. of the retentate in the region of. 2.8 mS/cm±15% corresponding to the conductivity of the 8 M urea buffer; and

(ii) a protein concentration in the region of 5 g/L after 2 successive final rinses of the ultrafilter with the diafiltration buffer.

The volume of the diafiltered concentrated eluate is about 3.7 L.

Ion-Exchange Chromatography

The column used possesses the following characteristics: Column (EMD TMAE Merck) having a diameter of 7 cm and a surface area of 38.5 cm²; gel height in the region of 18 cm; gel volume in the region of 700 ml.

The chromatography column is prepared as follows:

The gel is washed and then equilibrated at a flow rate in the region of 4 L/h with (i) 5 column volumes of ultrafiltered water, and then with (ii) 3 column volumes of 50 mM Tris buffer containing 8 M urea pH 7.5. The conductivity and pH values for the buffer at the outlet of the column should correspond to the following specifications for use: conductivity at 20° C.=2.8 ms/cm±15%, and pH=7.5±0.3.

The chromatography is carried out as follows:

The column is loaded by injecting the diafiltered eluate corresponding to 15-17 g of total proteins, at a flow rate of 100 mL/cm².h (4 L/h) and then rinsed with 50 mM Tris buffer containing 8 M Urea, pH 7.5, same flow rate, until there is a return to the base line (6 column volumes). The filtrate thus collected (about 4 L) contains the rAlpA protein 90±5% pure in the absence of endotoxins.

Sterile Filtration 0.2 μm and Bulk Storage of the Final Product

The purified rAlpA eluate is filtered on Supor Cap 100 0.8/0.2 μm type filter with a surface area of 1000 cm² in a laminar flow cabinet. The sterile filtered concentrated bulk volume is about 4 L.

The level of purity of the AlpA batch thus obtained is conventionally evaluated by SDS-PAGE, Coomassie blue staining and densitometric reading to be greater than 90%. The overall yield is estimated at about 50%.

1. A method for purifying Helicobacter adhesin-like protein A (AlpA), according to which (i) a preparation containing AlpA and 2.5 to 3.5 M guanidine is brought into contact, with a hydrophobic interaction chromatography material such that AlpA adsorbs onto the material and (ii) AlpA is eluted with a solution containing 3.5 to 4.5 M guanidine.

2. The method as claimed, in claim 1, in which the preparation used in point (i) contains 3 M guanidine.

3. The method as claimed in claim 2, in which the solution used in point (ii) contains 4 M guanidine.

4. A method as claimed in claim 1, 2 or 3, in which the preparation used in point (i) is a preparation derived from the culture of a gram-negative bacterium, especially E. coli, capable of expressing AlpA in recombinant form.

5. The method as claimed in claim 4, in which the preparation used in point (i) is obtained after breaking bacterial cells, washing and solubilizing the inclusion bodies.

6. The method as claimed in claim 5, in which the solubilization of the inclusion bodies is carried out in 5.5 to 6.5 M guanidine.

7. The method as claimed in claim 6, in which the solubilization of the inclusion bodies is carried out in 6 M guanidine.

8. The method as claimed in claim 5, 6 or 7, in which the preparation used in point (i) is obtained after breaking the bacterial cells, washing and solubilization of the inclusion bodies, precipitation with ammonium sulfate of the inclusion bodies solubilized and solubilization of the precipitate.

9. The method as claimed in claim 8, in which the solubilization of the inclusion bodies and/or of the precipitate is carried out in 5.5 to 6.5 M guanidine.

10. The method as claimed in claim 9, in which the solubilization of the precipitate is carried out in 6 M guanidine.

11. The method as claimed in one of claims 1 to 10, in which the hydrophobic interaction chromatography material comprises (i) a support which is Sepharose™ or Fractogel™ and (ii) a ligand which is butyl, octyl or phenyl.

12. The method as claimed in one of claims 1 to 11, in which the AlpA obtained after the elution carried out in point (ii) is suspended in 7.5 to 8.5 M urea and then purified on an ion-exchange chromatography material.

13. The method as claimed in claim 12, in which the suspension of AlpA in urea is carried out by diafiltration of the eluate obtained in point (ii), and optionally concentrated, against 6 to 10 volumes of 7.5 to 8.5 M urea.

14. The method as claimed in claim 12 or 13, in which the AlpA obtained after the elution carried out in point (ii) is suspended in 8 M urea.

15. The method as claimed in one of claims 11 to 14, in which the ion-exchange chromatography material comprises (i) a support which is Sepharose™ or Fractogel™ and (ii) a ligand which is a tertiary or quaternary amine.

16. A method for purifying AlpA according to which (i) a preparation containing AlpA and 7.5 to 8.5 M, preferably 8 M, urea is brought into contact with anion-exchange chromatography material such that the contaminants adsorb on this material and (ii) AlpA is recovered in the filtrate.

17. An AlpA protein characterized by a level of purity greater than or equal to 90% of, monomer, as measured by SDS-PAGE, Coomassie blue staining and densitometric reading.

18. A preparation containing at least 5 g of AlpA protein, the latter being characterized by a level of purity greater than or equal to 90% of monomer, as measured by SDS-PAGE, Coomassie blue staining and densitometric reading.
